(19) United States
(12) Patent Application Publication
(10) Pub. No.: US 2016/0149264 A1
(43) Pub. Date: May 26, 2016

(54) SPECIFIC FLUORINATED COMPOUNDS USED AS AN ORGANIC SOLVENT FOR LITHIUM SALTS

(71) Applicants: COMMISSARIAT À L'ÉNERGIE ATOMIQUE ET AUX ÉNERGIES ALTERNATIVES, Paris (FR); ECOLE NATIONALE SUPÉRIEURE DE CHIMIE DE MONTPELLIER, Montpellier Cedex 5 (FR)

(72) Inventors: Hervé GALIANO, LA VILLE AUX DAMES (FR); Stéphane Cadra, SAINT AVERTIN (FR); Bruno Ameduri, MONTPELLIER (FR); Vincent Besse, MILHAC DE NONTRON (FR); Ali Alaaeddine, MAJDAL SILIM (LB)

(21) Appl. No.: 14/903,135

(22) PCT Filed: Jul. 9, 2014
(86) PCT No.: PCT/EP14/64746

§ 371 Date: Jan. 6, 2016

(30) Foreign Application Priority Data
Jul. 10, 2013 (FR) 13 56790

(51) Int. Cl. H01M 10/0569; H01M 10/0525; C07D 327/04

(52) U.S. Cl. CPC H01M 10/0569; C07D 327/04; H01M 10/0525; H01M 2300/0028

(57) ABSTRACT

The disclosure relates to fluorinated compounds of following formula (I):

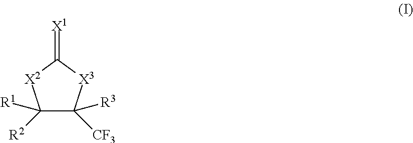

where:
X¹ is a sulfur atom;
X² and X³ are an oxygen atom or sulfur atom provided that, if one of said X² and X³ is a sulfur atom, the other of said X² and X³ is an oxygen atom and vice versa; and
R¹, R² and R³ are each independently a fluorine atom or hydrogen atom, provided that at least one of said R¹, R² and R³ is a fluorine atom. The disclosure further relates to use of these compounds as organic solvents of at least one lithium salt.

### TECHNICAL FIELD

The present invention pertains to specific fluorinated compounds, to their preparation method and to use thereof as solvents capable in particular of allowing the dissolving of lithium salts.

These compounds therefore naturally find application in the field of electrolytes and especially electrolytes intended to be a constituent part of lithium batteries.

Lithium batteries are of particular interest in sectors where battery life is an essential criterion such as in the area of computing, video, mobile telephony, transport e.g. electric vehicles, hybrid vehicles or further in the fields of medicine, space and microelectronics.

From a functional viewpoint, lithium batteries are based on the principle of lithium intercalation-deintercalation within the constituent materials of the electrodes of the battery's electrochemical cells.

More specifically, the reaction at the origin of current production (i.e. when the battery is in discharge mode) entails the transfer, via a lithium ion-conducting electrolyte, of lithium cations arriving from a negative electrode which come to be intercalated in the acceptor network of the positive electrode, whilst electrons derived from the reaction at the negative electrode will supply the external circuit to which the positive and negative electrodes are connected.

These electrolytes may be formed of a mixture comprising at least one organic solvent and at least one lithium salt to ensure the conducting of said lithium ions, which requires the lithium salt to be dissolved in said organic solvent.

At the current time the organic solvents used to ensure this function are conventionally carbonate solvents, such as ethylene carbonate, dimethyl carbonate, diethyl carbonate.

The inventors of the present invention have set out to develop novel compounds which have the following characteristics:
capable of easily dissolving lithium salts;
good electrochemical stability;
good thermal and chemical inertia; and
capable of reducing the flammability of the electrolytes in which they are incorporated.

### DISCLOSURE OF THE INVENTION

The invention is therefore directed towards fluorinated compounds of following formula (I):

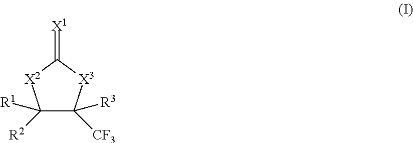

where:
X¹ is a sulfur atom;
X² and X³ are an oxygen atom or sulfur atom provided that if one of said X² and X³ is a sulfur atom, the other of said X² and X³ is an oxygen atom and vice versa; and
R¹, R² and R³ are each independently a fluorine atom or hydrogen atom, provided that one of said
R¹, R² and R³ is a fluorine atom.

To avoid any ambiguity it is finally more explicitly specified that when X² and X³ are an oxygen atom or sulfur atom with the condition that if one of said X² and X³ is a sulfur atom the other of said X² and X³ is an oxygen atom and vice versa (vice versa meaning that if one of said X² and X³ is an oxygen atom, the other of said X² and X³ is a sulfur atom), said compounds meeting this specificity can therefore be represented by one of following formulas (II) and (III):

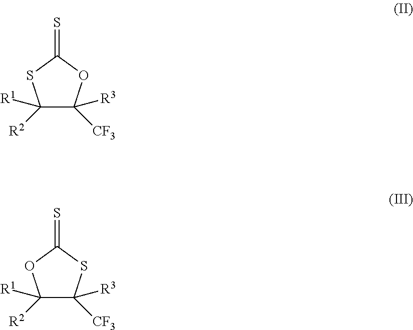

According to one particular embodiment, R¹, R² and R³ are each a fluorine atom in which case the compounds conforming to the invention meet following formula (IV):

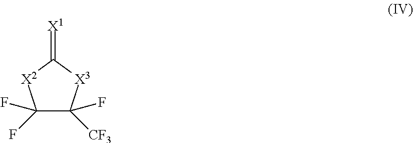

Specific examples of compounds coming under this definition are those meeting following formulas (V) and (VI):

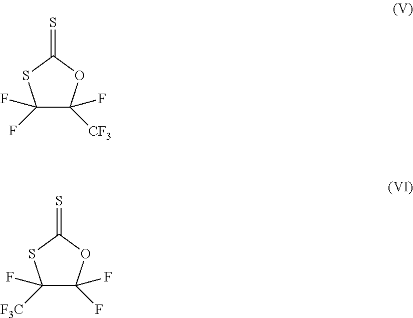 [SMILES: FC(F)(F)C1(F)OC(=S)SC1(F)F ; FC(F)(F)C1(F)SC(=S)OC1(F)F]

The fluorinated compounds of the invention can be prepared by implementing a method comprising a reaction step of an epoxide compound of following formula (VII):

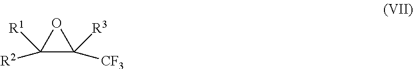

where R¹ to R³ meet the same definition as given above;

with a compound of following formula (VIII):

Z═C═ (VIII)

where Z is a sulfur atom.

One specific epoxide compound is hexafluoropropene oxide of following formula (IX):

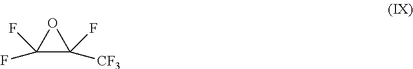

The present reaction is advantageously performed in the presence of a reaction catalyst capable of accelerating the chemical reaction between the epoxide compound of formula (VII) and the formula (VIII) compound. More specifically, the catalyst will interact with the oxygen atom of the epoxide compound to improve the reactivity thereof. After reaction of the epoxide compound of formula (VII) with the formula (VIII) compound, the catalyst is regenerated in its initial form.

The catalyst may advantageously be selected from among:
halogenated salts such as sodium chloride, sodium bromide, sodium iodide, potassium chloride, potassium bromide, potassium iodide, lithium chloride, lithium bromide;
organic salts such as sodium phenate, sodium acetate, sodium ethylate, sodium tert-butylate, potassium tert-butylate, tetrabutylammonium iodide, tetrabutylammonium bromide;
phosphonium salts such as phosphonium iodide;
cyclic organic compounds such as phenol, imidazole, pyrazole; and
the mixtures thereof.

If one of the compounds is in gaseous state under standard temperature and pressure conditions, the method of the invention can be carried out in a closed system such as an autoclave.

The reaction step can be conducted over a temperature range of 50° C. to 225° C. for a pressure range of 1 to 40 bars.

After the reaction step, the method may comprise a step to isolate the compound(s) from the reaction medium, this isolating step possibly being a succession of extraction, desiccation and evaporation operations of the organic solvent.

The compounds of the invention have particular properties such as sub-ambient melt temperature (e.g. lower than 0° C.), ability to separate ionic entities (due in particular to a dielectric constant which may be higher than 20) and chemical inertia against lithium salts.

They therefore not unexpectedly find application as organic solvent for at least one lithium salt, this organic solvent possibly being a constituent of an electrolyte comprising at least one lithium salt intended for a lithium battery.

The invention therefore also relates to:
the use of a fluorinated compound such as defined above as organic solvent of at least one lithium salt;
a composition, more specifically a liquid composition, which may be a lithium ion-conducting electrolyte, comprising at least one fluorinated compound such as defined above and at least one lithium salt; and
a lithium battery comprising at least one electrochemical cell comprising an electrolyte such as defined above arranged between a positive electrode and a negative electrode.

As examples, the lithium salt can be selected from the group formed by LiPF₆, LiClO₄, LiBF₄, LiAsF₆, LiCF₃SO₃, LiN(CF₃SO₂)₃, LiN(C₂F₅SO₂), lithium bis(trifluoromethylsulfonyl)imide (known under the abbreviation LiTFSI) LiN[SO₂CF₃]₂ and the mixtures thereof.

In the lithium battery the above-mentioned liquid electrolyte, in the electrochemical cells of lithium batteries, can be caused to impregnate a separator arranged between the positive electrode and the negative electrode of the electrochemical cell.

This separator may be in porous material such as a polymeric material able to receive the liquid electrolyte within its porosity.

The electrolyte is composed of at least one lithium salt and of at least one organic solvent which may be solely composed of one or more formula (I) compounds conforming to the invention, or it may further comprise at least one other aprotic solvent such as dimethyl carbonate, diethyl carbonate, ethyl and methyl carbonate, ethylene carbonate and propylene carbonate.

By positive electrode in the foregoing and in the remainder hereof is meant, as is conventional, the electrode which acts as cathode when the generator outputs current (i.e. when it is discharging) and which acts as anode when the generator is charging.

By negative electrode in the foregoing and in the remainder hereof is meant, as is conventional, the electrode which acts as anode when the generator outputs current (i.e. when it is discharging) and which acts as cathode when the generator is charging.

In general, the material of the negative electrode may be an active material which can be a carbon material such as graphite, or an oxide-type material of Li₄Ti₅O₁₂ type, said material possibly being associated with a polymer binder such as vinylidene polyfluoride, the resulting mixture possibly being deposited on a current collector in aluminium for example.

As for the material of the positive electrode, this may an active material of lithiated transition metal oxide type (the metal possibly being cobalt, nickel, manganese, iron for example), said material possibly being associated with a polymer binder such vinylidene polyfluoride, the resulting mixture possibly being deposited on a current collector in aluminium for example.

The invention is now described with reference to the following examples that are non-limiting and given by way of indication.

### DETAILED DESCRIPTION OF PARTICULAR EMBODIMENTS

### Example 1

The following example illustrates the preparation of a compound conforming to the invention (4,5,5-trifluoro-4-(trifluoromethyl)-1,3-oxathiolan-2-thione) according to the following reaction scheme:

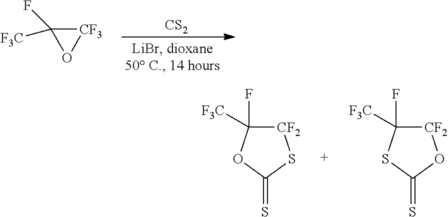

Since hexafluoropropene oxide is gaseous, the reaction is performed in a 100 mL Parr Hastelloy autoclave equipped with a manometer, rupture disc and gas inlet and release valves. An electronic device is used to control both agitation and heating of the autoclave.

209 mg (2.4 mmol) of lithium bromide were placed in the autoclave and the device pressurized to 30 bars nitrogen for 1 hour to check imperviousness. The device was considered to be gas-tight if no decrease in pressure was ascertained during an observation time of one hour. The synthesis protocol could then be continued. The nitrogen was evacuated and the autoclave placed under a vacuum for 30 minutes. 30 mL of dioxane and 11 g (0.144 mol) of carbon disulfide were then added. The autoclave was cooled to −30° C. by immersion in a mixture of acetone and liquid nitrogen after which 8 g (0.048 mol) of hexafluoropropylene oxide were added. The autoclave was then heated up to 50° C. Throughout the reaction, the pressure measured in the autoclave reached 12 bars then decreased and was held at 8 bars at 50° C. 14 hours after initiation of the reaction the autoclave was cooled by immersion in an ice bath for 30 minutes. The reactor was then degassed and the reaction residue collected.

Finally the residue was washed with 50 mL water and extracted 3 times with dichloromethane. The different organic phases were combined, dried over sodium sulfate, filtered and evaporated. A translucent oil was obtained.

Collection of the compound gave a yield of 76%.

The compound was respectively subjected to analysis by ¹⁹F NMR, 400 MHz (CDCl₃) and ¹³C NMR, 400 MHz (CDCl₃).

The ¹⁹F NMR spectrum of the compound gave the following signals corresponding to the C—F fluorine of the cyclic structure;
δ (ppm): −132.81; −130.14; −122.82 corresponding to the C—F fluorine of the cyclic structure;
δ (ppm): −89.04; −87.38; −83.90; −78.48 corresponding to the —CF₂ fluorines of the cyclic structure;
δ (ppm): −82.09 corresponding to the fluorines carried by the —CF₃ group.

The RMN ¹³C NMR spectrum of the compound gave the following signals:
δ (ppm):101.87 to 108.87 corresponding to a doublet of quadruplets of a coupling constant for the C—F group of the cyclic structure;
δ (ppm): multiplets of 113.62 to 125.49 corresponding to the —CF₂ carbon of the cyclic structure;
δ (ppm): 159.08 and 162.45 corresponding to the thiocarbonyls of both isomers.

1. A fluorinated compound of following formula (I): 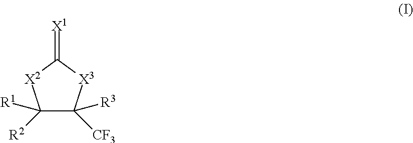
where:
X^{l} is a sulfur atom;
X² and X³ are an oxygen atom or sulfur atom provided that if one of said X² and X³ is a sulfur atom, the other of said X² and X³ is an oxygen atom and vice versa; and
R¹, R² and R³ are each independently a fluorine atom or hydrogen atom provided that at least one of said R¹R² and R³ is a fluorine atom.

2. The fluorinated compound according to claim 1 wherein R¹, R² and R³ are each a fluorine atom, having the following formula (IV):

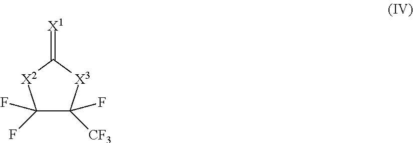

3. The fluorinated compound according to claim which corresponds to a compound of one of the following formulas:

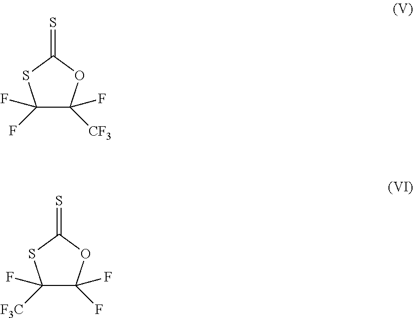 [SMILES: FC(F)(F)C1(F)OC(=S)SC1(F)F ; FC(F)(F)C1(F)SC(=S)OC1(F)F]

4. A method of forming a composition comprising combining at least one lithium salt and a fluorinated compound according to claim 1 as organic solvent of the at least one lithium salt.

5. A composition comprising at least one fluorinated compound as defined in claim 1 and at least one lithium salt.

6. The composition according to claim 5, wherein the composition is a lithium ion-conducting electrolyte.

7. A lithium battery comprising at least one electrochemical cell comprising an electrolyte as defined in claim 6 arranged between a positive electrode and a negative electrode.

8. A composition comprising at least one fluorinated compound as defined in claim 2 and at least one lithium salt.

9. A composition comprising at least one fluorinated compound as defined in claim 3 and at least one lithium salt.
